(19) United States
(12) Patent Application Publication
(10) Pub. No.: US 2003/0005601 A1
(43) Pub. Date: Jan. 9, 2003

(54) Corrective appliance mounted to footwear for correcting and preventing deformity of a foot

(76) Inventor: Iwao Kasahara, Yokohama-shi (JP)

Correspondence Address: JORDAN AND HAMBURG LLP, 122 EAST 42ND STREET, SUITE 4000, NEW YORK, NY, 10168, US

(21) Appl. No.: 10/217,996

(22) Filed: Aug. 13, 2002

Related U.S. Application Data
(63) Continuation of application No. 09/544,252, filed on Apr. 5, 2000.

(30) Foreign Application Priority Data
Apr. 7, 1999 (JP) 11-136135
May 12, 1999 (JP) 11-168720
Mar. 22, 2000 (JP) 2000-079681
Mar. 31, 2000 (JP) 2000-096208

(51) Int. Cl. A43B 7/14; A61F 5/14

(52) U.S. Cl. 36/88; 36/140

(57) ABSTRACT

There is provided a corrective appliance mounted to a footwear so as to correct the soles as a whole that are deformed or deviated as a whole by cause of Hallux Vulgus and etc. The corrective appliance comprises a first pressing member for pressing the base of a first toe or phalanx and the first metatarsal caput as protruding from the corrective position, and a second pressing member for pressing a lowermost proximal end of a fifth metatarsal as protruding from the corrective position so as to narrow a partial width between walls on the both sides of the footwear. In the case where the corrective appliance is used for footwear such as shoes or sandals, the first and second pressing or reinforcing members are provided on the side inside or outside of the footwear. In the case where the corrective appliance is used for footwear such as hoses, the first and second pressing or reinforcing members are provided on a looped elastic band or bands having elasticity larger than other cloth of the hoses. Furthermore, in the case where the corrective appliance is used for footwear such as hoses, it increases effectiveness of correcting the deformed or deviated a sole or a foot by additionally providing one or both of a first protuberant member for correcting a widthwise arch of the sole, and a second protuberant member for correcting a lengthwise arch of the sole.

### FIELD OF THE INVENTION

The present invention relates to a corrective appliance for correcting or preventing deformity or deviation of a foot, which is used with footwear such as shoes, sandals and socks, and more particularly to a corrective appliance for correcting or preventing an outward deformity or excessive deviation of a foot caused by hallux valgus so that a correct walking is made with a balanced sole.

### BACKGROUND OF THE INVENTION

In recent years, since human beings had a little opportunity for walking with in accordance with the advancement in science technology, their foots weakened their functions. For this reason, they acquired disorders such as hallux valgus, and walking that their toes touched with the ground. Such the disorders increased, irrespective of progress of medical sciences. The disorders involves unbalanced soles due to repeated wears of high heels and sandals.

When walking is repeatedly made with injured foots and unbalanced soles, human beings are suffered from shock and torsional waves repeatedly transferred to their body. Generally, the shock and torsional waves are naturally produced, though balanced walking is made. However, the shock and torsional waves of the unbalanced walking is magnified into several times than naturally produced one. Therefore, the shock and torsional waves caused by Hallux Valgus, and the unbalanced walking that the toes can't touch with the ground, become excessive shock and deviation to cause the human body to be suffered from the injurious stresses. The injurious stresses are naturally accumulated in the human body little by little so that the human body experiences unidentified pain (chronic disease) whose cause is unknown, and no good condition.

Most of Hallux valgus are caused by repeatedly wearing fashionable or stylish shoes such as high heels and sandals having a pointed shape which is situated at a toe of a foot. Therefore, the hallux valgus one of disorder of which many women experience. In these days, fashionable or stylish shoes are also desired for male shoes, and number of men with hallux valgus is on the increase, because of that many male shoes are apt to be formed in a narrower shape. For this reason, shoes on the market are also produced by wider shape than conventional ones. However, according to a research by a doctor, it is explained that a wearer's foot suffered from Hallux Vulgus is put in shoes with large width but the symptom of her or his foot become worse, because her or his foot is easily widened, and unbalanced.

Symptom of hallux valgus is, as shown in FIG. 1, deviation of a first finger 1 to a second finger of a foot, and an abnormal inward protrusion with respect to the human body of a supporting point at an enlargement (caput) 2MD of a first metatarsal 2 on the side of its distal end, as indicated by the arrow A, whereby the caput 2MD of the first metatarsal 2 is inwardly deviated so that a lowermost proximal end 3ML of a fifth metatarsal 3 at opposite position to the caput 2MD of the first metatarsal 2 (point of application 4d) is also outwardly deviated, as indicated by the arrow B. This function can best be explained by imagining the principle of lever.

When a wearer puts her or his foot into a shoe with a pointed shape or narrowed width, her or his first finger 1 is displaced toward a second finger, as it is .tight by the inner surface of the shoe. The first toe 1 is rotated about the ball thereof, and the caput 2MD of the first metatarsal 2 is displaced inward of the wearer's foot. That is, the displacement of the first toe 1 provides a leverage, because the first toe 1 is (a point 4c where force is applied), and an enlargement (caput) 2MD of the first metatarsal 2 is (a supporting point) 4. The force transmits inward of the wearer's foot from the first toe 1. The caput 2MD of the first metatarsal 2 goes inward, while the force transmits to the lowermost proximal end 3ML of the fifth metatarsal 3 as (point of application) 4e.

The wearer feels a pain in the enlargement (caput) 2MD of the first metatarsal 2, because the enlargement (caput) 2MD of the first metatarsal 2 is brought into contact with the inside surface of the shoe. Therefore, the wearer tries to separate the enlargement (caput) 2MD of the first metatarsal 2 from the inside surface of the shoe during her or his walking, the force naturally toward the lowermost proximal end 3ML of the fifth metatarsal 3 which is in the area where the inside surface of shoe is not brought into contact therewith. As a result, force caused by the first toe 1 transmits to the lowermost portion 3ML of a fifth metatarsal 3 at the proximal end thereof, and the lowermost portion of a the fifth metatarsal at its proximal end become (the point of application 4e). This causes unbalanced walks so that shock and torsional waves are generated. Finally, her or his body as a whole experiences stresses to cause her to feel dizzy, chronic pain, and no good condition.

Conventional invention of corrective appliances is disclosed in Japanese unexamined patent publication No. Heisei (Tokkaihei) 6-62906, Japanese unexamined patent publication No. Heisei (Tokkaihei) 7-308334, and Japanese unexamined patent publication No. Heisei (Tokkaihei) 8-134477. Japanese unexamined patent publication No. Heisei (Tokkaihei) 6-62906 shows a corrective appliances for Hallux Valgus for adjusting a width between a first finger and a second finger in angle so as to pull back a first metatarsal with outward deviation caused by Haliux Valgus. Japanese unexamined patent publication No. Heisei (Tokkaihei) 7-308334 shows an appliance for preventing exhaustion of a foot with Hallux Valgus. The appliance comprises an wider elastic band which is wound from a sole to an instep of a foot at a position as a whole of a metatarsal., and a thin band which is wound on a first toe so as to pull back a first metatarsal with outward deviation, the fine band being sewn on a substantially center portion of the wider elastic band and extending from the center portion to the first toe. Japanese unexamined patent publication No. Heisei (Tokkaihei) 8-131477 shows a tape for correcting a foot having Hallux Valgus. The corrective tape is directly adhered to a location which inwardly extends from an outer edge to an instep at a metatarsal of a foot, and to a location which extends from a branched portion of the inwardly extending location along at a side edge at the metatarsal of the foot so that the deviated foot is inwardly corrected from a first toe to a first metatarsal.

Both of Japanese unexamined patent publication Nos. Heisei (Tokkaihei) 6-62906 and 7-308334 are directed to a spread width between the first and second toes of the foot with Hallux valgus. Such the corrective appliance can temporarily correct the deviation of the first toe and metatarsal. However, the width as a whole of the foot would be-widened. Therefore, when the wearer wears put her or his foots in the high heel or sandal, she or he would be suffered from more problems such as a tightness and a poor blood circulation. The corrective tape disclosed in Japanese patent publication No. Heisei 8-131477 sticks to a foot of the wearer, since it is directly adhered to a skin of the foot with Hallux Valgus of the wearer. The wearer would feel uncomfortableness, when she walks, because the corrective tape adheres to her foot. The adhesive of the tape sticks to the skin of the foot, when the tape is peeled from the foot of the wearer or when the tape is adhered to the foot of the wearer. Therefore, in spite of the tape for correcting Hallux Valgus, the tape caused disagreeable feeling to the foot of wearer.

Furthermore, U.S. Pat. No. 5,607,756 discloses a sprint, in which Hallux Valgus of the wearer is corrected by pushing up the arch corresponding the metatarsal at the back side of the foot, and inwardly pressing both sides of the metatarsal of the foot. The sprint does not widen distance between the first finger and second finger. of the wearer.

When the sprint is used, outward deviation of the foot due to Hallux Valgus would be temporarily corrected by the combination of the pushed arch and the inwardly pressed metatarsal. However, the wearer is not always suffered from Hallux Valgus of both foots. For the reason, the wearer often has to use one of the sprints with for correcting one foot of Hallux Valgus, when he walks. As a result, since only one foot of the wearer is unnaturally corrected, the wearer can not walk with balanced state.

Moreover, Hallux Valgus was also corrected by the sole of the shoe or an insole with a corrective form. When the wearer walks with use of such the shoe or the insole, she or he would feel deviation of his arch from the corrective position for the shoe or the insole. In addition, when the foot of the wearer sweats, the wearer feels that her sole gets sweaty at the corrective portion or slides from the corrective portion. Such the corrective appliances will not fit the wearer. Further moreover, when the wearer walks with use of such the corrective appliances, she is suffered from wrinkles formed at a position corresponding to the corrective potion of the corrective appliance on her or his sox. Accordingly, the wearer will cause stress.

U.S. Pat. No. 5,792,093 discloses an corrective appliance used with a sox for correcting Hallux Valgus of a foot. The corrective appliance includes a Y shaped acupressure projection. The Y shaped acupressure projection is fixed on a surface inside of a sox such that desired portions of an instep of the foot is suitably pressed by the projection.

### SUMMARY OF THE INVENTION

A first objective of the present invention is to provide a corrective appliance having a simple construction, which enables correcting deformity or deviation of a foot with a simple construction

A second objective of the present invention is to provide a corrective appliance, which enables correcting the deformed or deviated foot without use of footwear having a toe formed in large width so as to widen a width between first and second toes of a foot.

A third objective of the present invention is to provide a corrective appliance for Hallux valgus, which has a construction allowing the footwear to be narrowed at the portions where the caput of the first metatarsal (thebase of the first toe) supporting point) and the lowermost proximal end of the fifth metatarsal is positioned, such that the force applied to the first toe doesn't transmit outward with respect to the human body. The correction is to apply a dynamic principle such that the caput of the first metatarsal (the base of the first toe) supporting point) and the lowermost proximal end of the fifth metatarsal (point of application) are pressed whereby the toes are opened and the foot is corrected.

In order to attain the above objective, the present invention provides a corrective appliance for Hallux Valgus used with footwear, the appliance comprising a first press member for pressing the first toe or pahalax and the caput of the first metatarsal that are in the area protruding form the correct position, and a second press member for pressing the lowermost proximal end of the fifth metatarsal that is in the area protruding from the correct position.

In the case where the corrective appliance is used for footwear such as hoses, it increases effectiveness of correcting the deformed or deviated sole or foot by additionally providing a first protuberant member for correcting a widthwise arch of the sole, and a second protuberant member for correcting a lengthwise arch of the sole.

### BRIEF DESCRIPTION OF THE DRAWINGS

FIG. 1 is a view illustrating a abnormally deformed foot.

FIG. 2 is a plan view illustrating a skeleton in a normal foot.

FIG. 3A is a sectional plan view in use of the shoes according to first embodiment of a corrective appliance as applied to a shoe.

FIG. 3B is a side view illustrating the shoe with the corrective appliance as seen from the side of the first finger.

FIG. 3C is a side view illustrating the shoes with the corrective appliance as seen from the side of the fifth finger.

FIG. 4 is a plan view illustrating a corrective appliance as applied to a sandal in accordance with third embodiment of the present invention.

FIG. 5 is a plan view illustrating a corrective appliance as applied to a Japanese sandal in accordance with fourth embodiment of the present invention.

FIG. 6 is a plan view illustrating a corrective appliance as applied to a sandal in accordance with fifth embodiment of the present invention.

FIG. 7 is a plan view illustrating a corrective appliance as applied to an insole for shoes in accordance with sixth embodiment of the present invention.

FIG. 8A is a plan view illustrating elements used for a corrective appliance as applied to a hose in accordance with seventh embodiment of the present invention

FIG. 8B is a perspective view illustrating a corrective appliance as applied to the hose in accordance with seventh embodiment.

FIG. 9 is an external oblique view illustrating a corrective appliance as applied to a pair of knee socks in accordance with eighth embodiment of the present invention.

FIG. 10 is an external oblique view illustrating a corrective appliance as applied to a pair of panty hose in accordance with ninth embodiment of the present invention.

FIG. 11 is an external oblique view illustrating a corrective appliance as applied to a hose having five inserts for finger in accordance with tenth embodiment of the present invention.

FIG. 12 is an external oblique view as applied to a hose in accordance with eleventh embodiment of the present invention.

FIG. 13A is a plan view illustrating a sole of a foot in the correct state.

FIG. 13B is a view of the sole looking from the front side of FIG. 13A.

FIG. 14 is a perspective view illustrating the corrective appliance having protuberant members.

FIG. 15 is an external oblique view illustrating the corrective appliance having protuberant members.

FIG. 16 is an external oblique view depicting action in the corrective appliance.

FIG. 17 is a perspective top view depicting a corrective appliance partially formed by a side wall of a shoe.

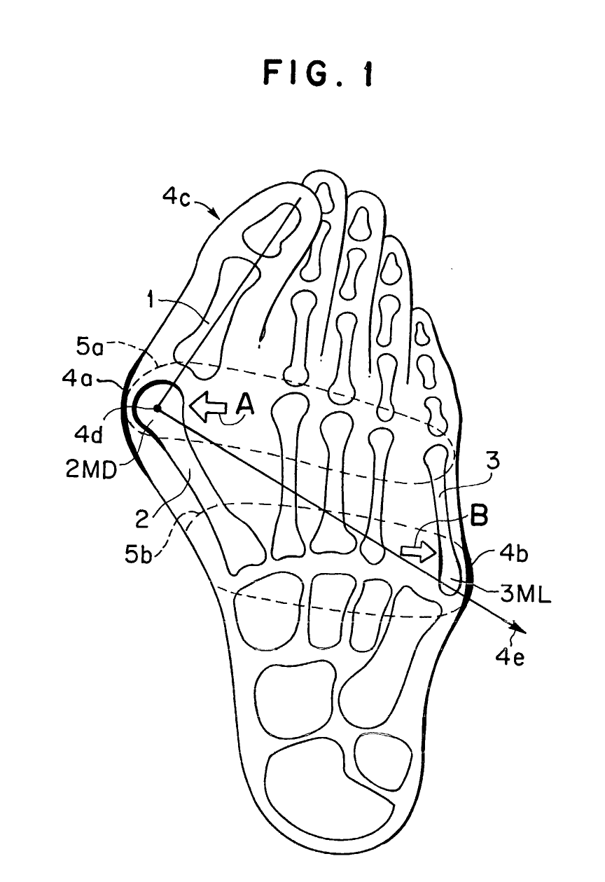

### PREFERRED EMBODIMENTS OF THE INVENTION

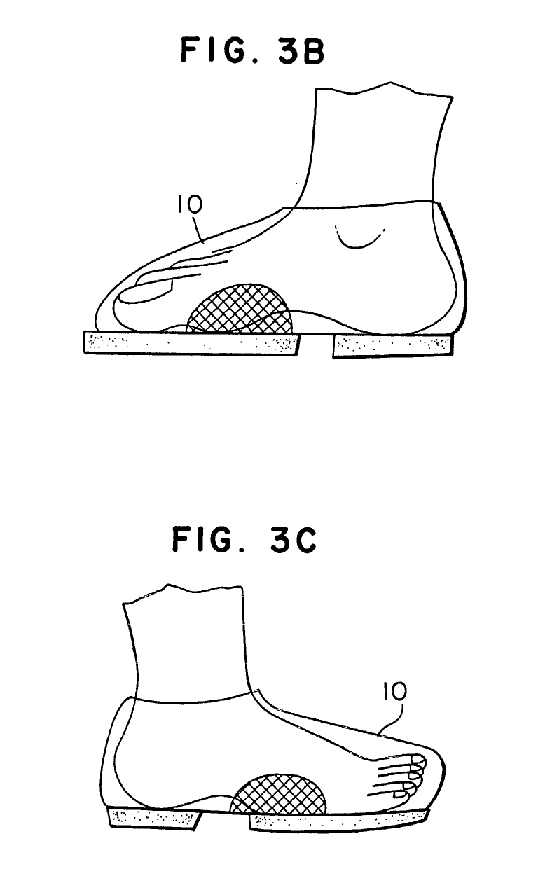

The description will be made in detail of the embodiments of the present invention by reference to the drawings. First embodiment of the present invention is indicated by FIGS. 3A to 3C. In the illustrated embodiment, the invention is applied to a shoe. Referring to FIGS. 3A to 3C, a wearer's foot is put into a shoe 10. FIG. 3A illustrates a state of seeing the embodiment according to the present invention from the above. The distal end of the shoe 10 takes up a position where the first to fifth metatarsals occupy. In the illustrated shoe 10, a pressing member 11 and a pressing member 12 are attached at the flank inside of the shoe 10 in face to face each other.

The first and second pressing members each have the thickness, the top surface, and the back surface. The top surfaces of the pressing members contact with the inner surface of the shoe 10, and the back surfaces of the pressing members contact with the foot.

Thus, the pressing member 11 serves to press, as indicated by the arrow C, against a lowermost portion of the fifth metatarsal 3 at the proximal end thereof that is in the area 4b protruding from the correct position by cause of Digitus Varus Minimi, and the pressing member 12 serves to press, as indicated by the arrow D, against the first toe or phalange and the caput of the first metatarsal that are in the area 4a protruding from the correct position by cause of Hallux Vulgus. The pressing members 11, 12 may be replaced by reinforcing members mounted at the flank outside of the shoe 10.

Accordingly, when a wearer's foot is fit in the shoe 10, the lowermost proximal end of the fifth metatarsal 3 that is in the area 4b protruding form the correct position by cause of Digitus Varus Minimi is pressed by the pressing member 11, as shown in FIG. 3C, and the first toe or phalange 1 and the first metatarsal 2 that are in the area 4a protruding from the correct position by cause of Hallux Vulgus is pressed by the pressing member 12, as shown in FIG. 3B.

FIG. 17 is a drawing illustrating second embodiment according to the present invention. A shoe 101 narrows its distance “a” between its side wall in contact with the caput 2MD of the first metatarsal 2 protruding from its correct position by cause of Hallux Valgus, and a side wall disposed in the shoe 101 and on the opposite side of the first metatarsal 2, and its distance “b” between its side wall 103 in contact with the lowermost proximal end 3ML of the fifth metatarsal 3 protruding from its correct position by cause of Digitus Varus Minimi, and a side wall disposed on the opposite side of the fifth metatarsal 3 in the shoe 101. In the shoe 101, its wall is partially thickened to narrow the distances “a” and “b” whereby the ball of the first toe or the phalange 1 and the lowermost proximal end 3ML of the fifth metatarsal 3 protruding from their correct position is pressed, as indicated by the arrows C, D. That is, the wall of the shoe 101 functions to correct the deviated foot similar to the pressing members 11, 12.

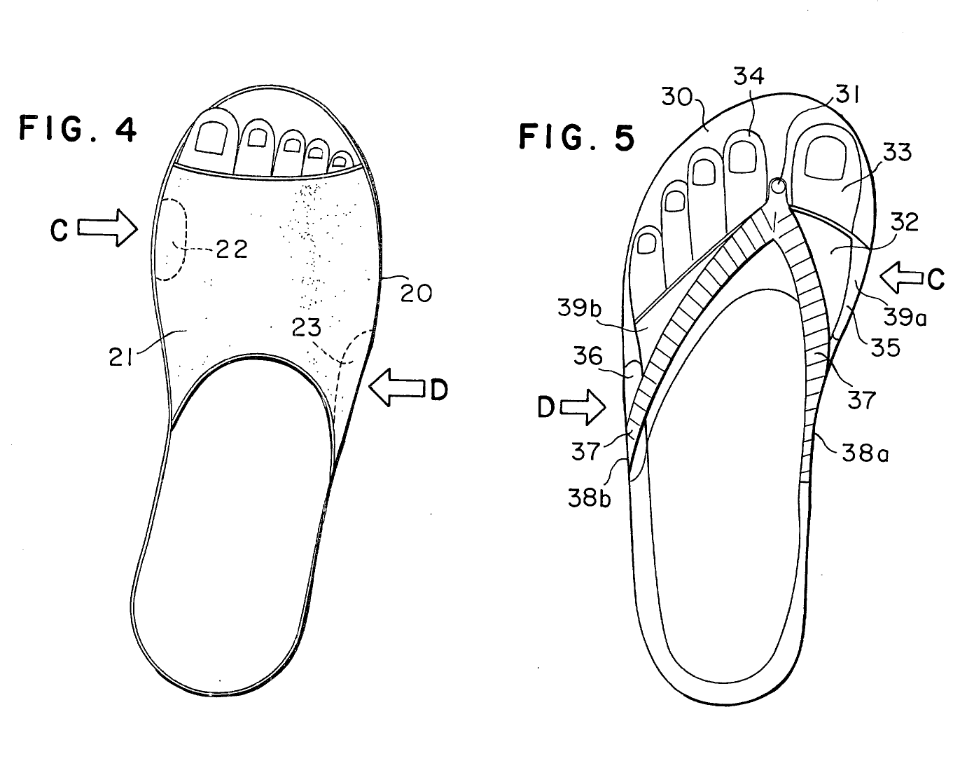

FIG. 4 is a drawing illustrating third embodiment according to the present invention. Referring to FIG. 4, a sandal 20 has a wide strap 21 fixed on both sides of the top surface thereof. The wide strap 21 has a first pressing member 22 mounted on the inner surface thereof for pressing, as indicated by the arrow C, the ball of the first toe or phalange 1 and the first metatarsal caput 2MD that are in the area 4a protruding form the correct position, arid a second pressing member 23 for pressing, as indicted by the arrow D, the lowermost proximal portion 3ML of the fifth metatarsal 3 that is in the area 4b protruding from the correct position.

FIG. 5 is a drawing illustrating fourth embodiment of the present invention. Referring to FIG. 5, there is shown a Japanese sandal 30 having a clog thong 31 between a first toe 33 and a second toe 34 near the distal end thereof, and a strap 37 extending from the clog thong to both of side edges 38a, 38b. The Japanese sandal 30 has a band 32 disposed below the strap 37. The band 32 extends from the area between the protuberant toe or phalange and the first metatarsal to the lowermost proximal end 3ML of the fifth metatarsal 3 thereof in curve path. In the Japanese sandal 30, the band 32 is fixed to the clog thong 31, and both side edges 38a, 38b thereof Therefore, when a wearer's first and second toes 33 and 34 are positioned on both sides of the clog thong 31 in the Japanese sandal 30, the area where the wearer's the balls of toes and metatarsals at its proximal end are positioned, is held or covered by the band 32. The band 32 has a pressing member 35 disposed on the inner surface thereof for pressing, as indicted by the arrow C, the first toe or phalange 1 and the first metatarsal 2 that are in the area 4a protruding from the correct position by cause of Hallux Vulgus, and a pressing member 36 disposed on the inner surface thereof for pressing, as indicted by the arrow D, the lowermost proximal end 3ML of the fifth metatarsal 3 that is in the area 4b protruding form the correct position by cause of Digitus Varus Minimi.

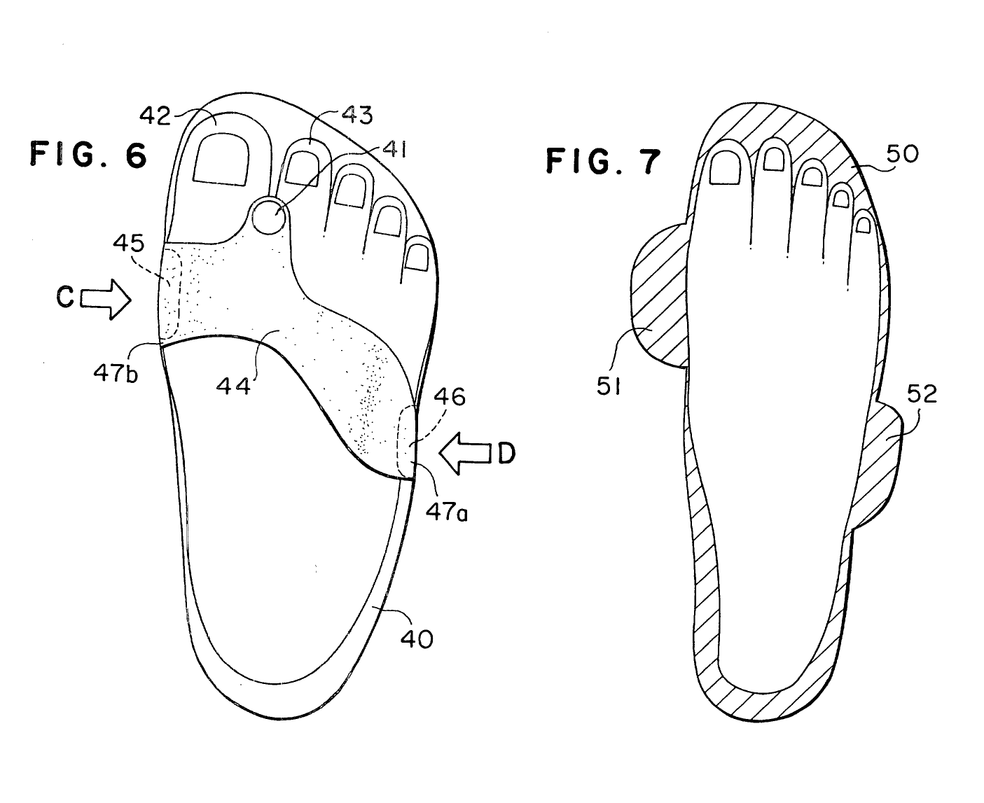

FIG. 6 is a drawing illustrating fifth embodiment of the present invention. Referring to FIG. 6, there is shown a Japanese sandal 40 having a clog thong 31 between a first toe 42 and a second toe 43 near its distal end, and a strap 44 extending from the clog thong 41 to both of side edges. The strap 44 has a pressing member 45 disposed on the inner surface thereof for pressing the first toe or phalange 1 and the first metatarsal 2 that are in the area 4a protruding from the correct position by cause of Hallux Vulgus, and a pressing member 46 disposed on the inner surface thereof for pressing the protuberant proximal end of the fifth metatarsal at the lowermost portion that is in the area 4b protruding form the correct position by cause of Digitus Varus Minimi. Therefore, when a wearer's first toe 42 and second toe 43 are positioned on both sides of the clog thong 31, respectively, protuberant areas of her or his foot is pressed by the press members 45, 46, respectively.

FIG. 7 is a drawing illustrating a sixth embodiment of the present invention. Referring to FIG. 7, there is shown an insole 50, in which a pressing member 51 for pressing the first toe or phalange 1 and the first metatarsal caput 2MD that are in the area 4a protruding form the correct position by cause of Hallux Vulgus, and a presssing member 52 for pressing the lowermost proximal end 3ML of the fifth metatarsal 3 that is in the area 4b protruding from the correct position by cause of Digitus Varus Minimi, are provided. The pressing member 51 is disposed on the side of the insole 50 along the ball of the fist toe, and the first metatarsal caput, and the pressing member 52 is disposed on the side of the insole 50 along the fifth metatarsal. Both of the pressing members 51, 52 are integrally provided with the insole 50, and comprises a cushion. Therefore, when the insole 50 is mounted within a shoe, the faces on pressing members 51, 52 are pressed into contact with a wearer's foot by an inner surface of the shoe so that the protuberant portions of the foot is pressed.

The description will be made below in relationship with embodiments of the present invention as applied to a pair of hose.

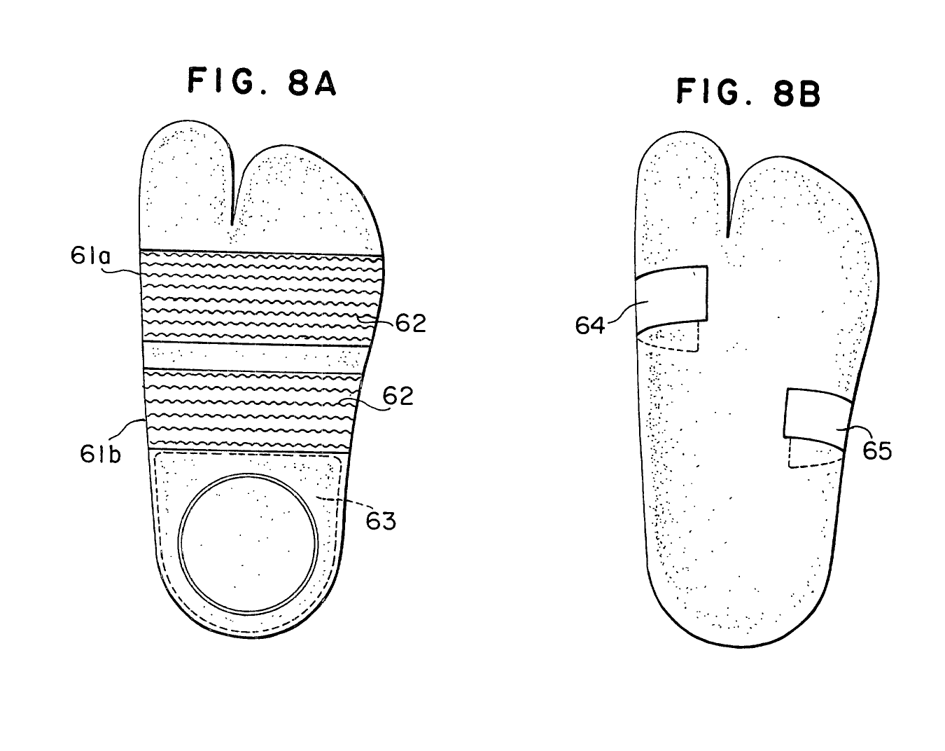

FIG. 8A is a drawing illustrating a seventh embodiment of the present invention. Referring to FIG. 8A, there is shown a hose having bands 61a, 61b, which form a loop covering an instep, and a sole. The distal end of the first metatarsal 2, and the proximal end of the fifth metatarsal 3 are covered with the bands 61a, 61b, respectively. The bands 61a, 61b have an elastic material or yarn knitted thereinto that is of larger resilience than other parts. Furthermore, the hose has a cushioning plate 63 provided on the inner surface of the hose at a position corresponding to a heel. No need to inevitably provide the cushioning plate 63.

FIG. 8B is a perspective view of the hose as shown in FIG. 8A. The hose has a pressing (or reinforcing) member 64 provided on the inside surface thereof so that the first toe or phalange 1 and the first metatarsal caput 2MD protruding from the correct position is brought into contact therewith, and a pressing (or reinforcing) member 65 provided on the inside surface thereof so that the lowermost proximal end of the fifth metatarsal which is in the area 4b protruding from the correct position. It will be understood that the pressing (reinforcing) members 64, 65 are disposed in overlapping relation with the bands 61a, 61b with the elastic material or yarn knitted therein, respectively.

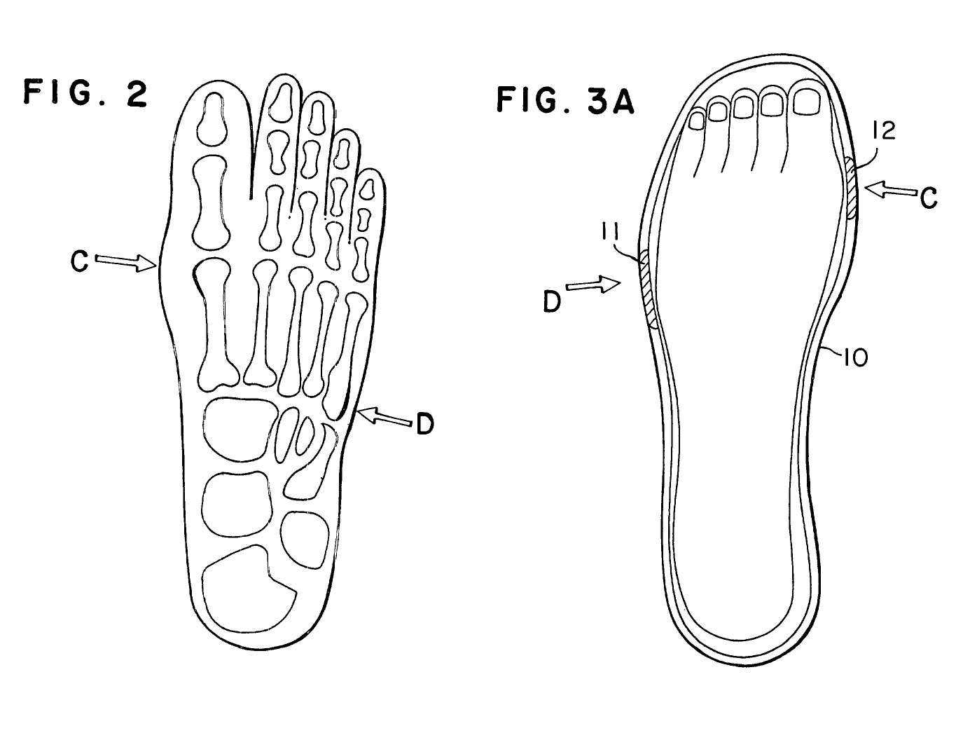

Thus, when the wearer put her or his foot into the hose, the area extending from the balls of the toes or phalanges at its proximal end to the medial portion of the metatarsals is tight by the first band 61a, and the area extending from the medal portion of the metatarsals to the balls of the metatarsals at the proximal end is tight by the second band 61b, which is apart form the first band 61a. Furthermore, the first pressing (reinforcing) member 64 presses the first toe or phalange and the first metatarsal caput that are in the area 4a protruding form the correct position by cause of Hallux Vulgus, and the second pressing (or reinforcing) member 65 presses the lowermost proximal end of the fifth metatarsal 3 that is in the area 4b protruding from the correct position by cause of Digitus Varus Midimi. Thereby, the deformed foot of the wearer will be corrected as shown in FIG. 2.

The corrective appliance according to the present invention is applied to various kinds of hoses.

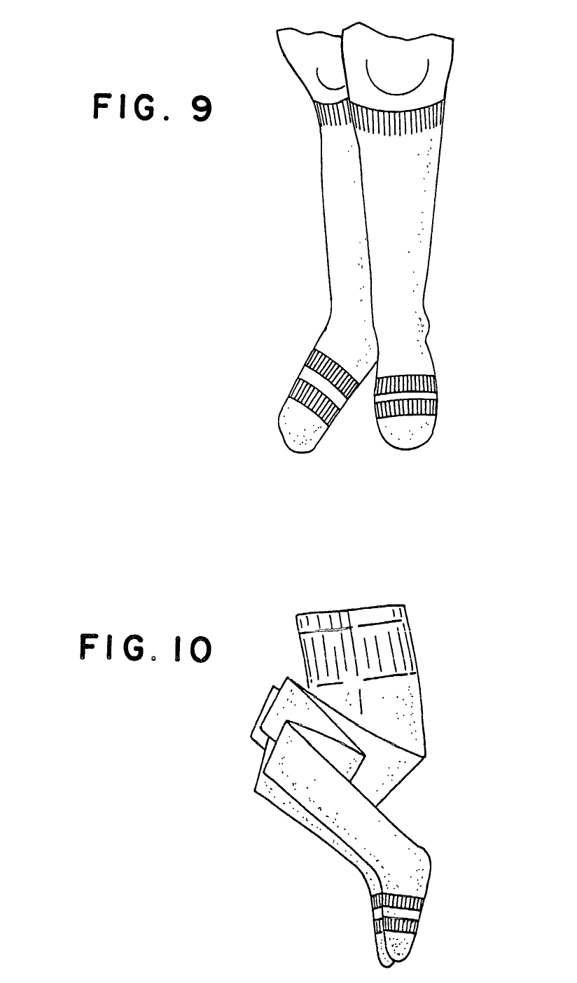

FIG. 9 is a schematic illustration of the present invention as applied to a pair of knee socks in accordance with eighth embodiment of the present invention.

FIG. 10 is a schematic illustration of the present invention as applied to a pair of panty hose in accordance with ninth embodiment of the present invention.

FIG. 11 is a schematic illustration of the present invention as applied to a hose forming five inserts for inserting five toes in accordance with tenth embodiment of the present invention.

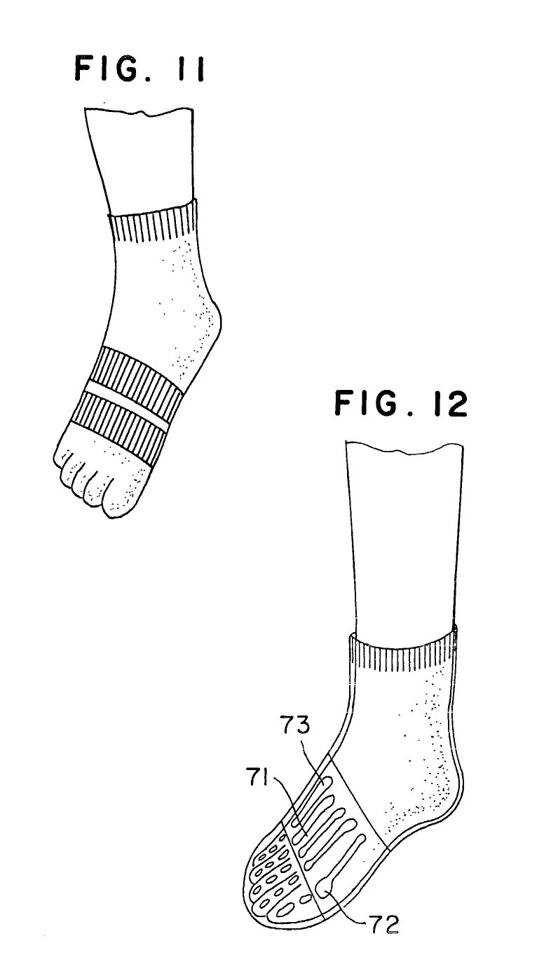

FIG. 12 is a drawing illustrating eleventh embodiment of the present invention. Referring to FIG. 12, there is shown a sox having an elastic band 71 capable of covering and tightening the area extending form the distal end to the proximal end of the metatarsals. The strip 71 has a pressing member 64 for pressing the first toe or phalange 1 and the first metatarsal caput 2MD that are in the area 4a protruding from the correct position, and a pressing member 65 for pressing the lowermost proximal end of the fifth metatarsal 3ML that are in the area 4b protruding from the correct position. Thus, when a wearer put her or his foot into the sock, the distal end 72 of the first metatarsal 2 and the proximal end 73 of the fifth metatarsal 3 that are in the area 4b protruding from the correct position, are pressed by a single elastic band 71. When the corrective appliance according to the present invention is used for a hose 90, it increases effectiveness of correcting the deformed or deviated foot by providing protuberant members 91 and 92 as well as the pressing member 64 and 65, as shown in FIG. 14.

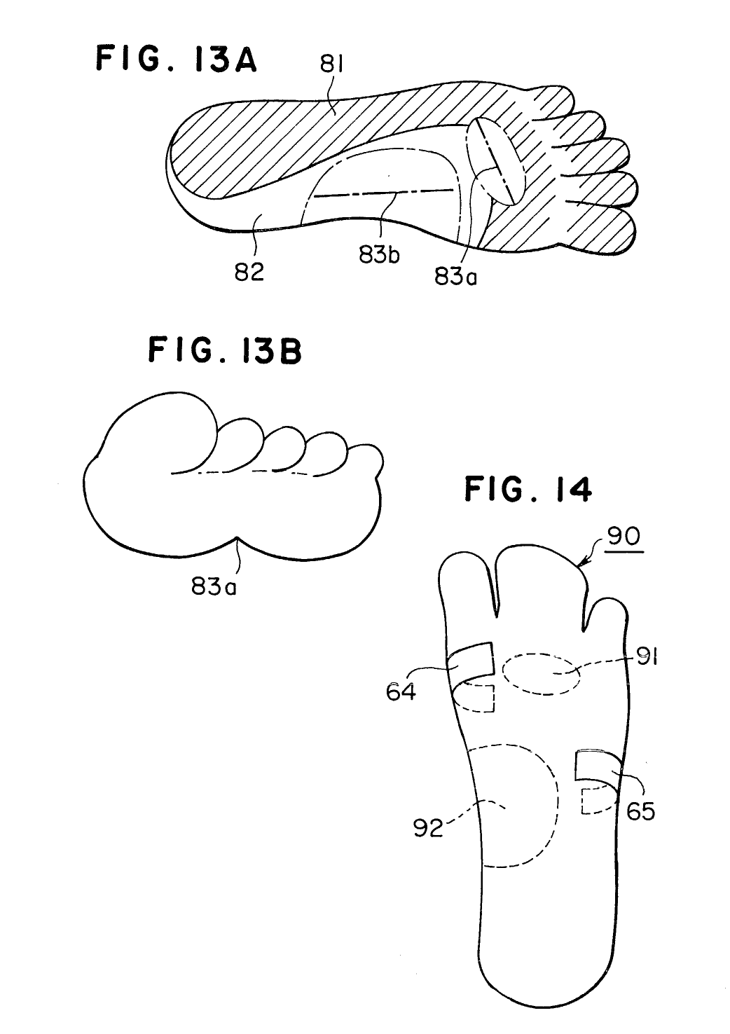

FIG. 13A is a plan view illustrating a sole of a foot, which is in the correct state. The sole has a slanted area 81 which is brought into contact with the ground, and a sunken area 82 which is difficult to touch with the ground. The sunken area 82 has a first recess 83a forming widthwise arch-shape of the sole, and a second recess 83b forming lengthwise arch-shape of the sole.

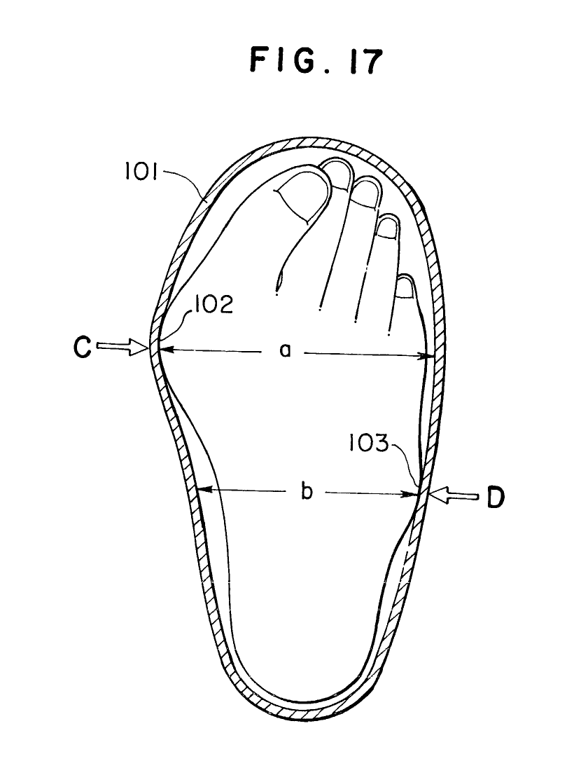

The first protuberant member 91 has a top surface formed in the shape of the first recess 83a, as shown in FIG. 13B, and the second protuberant member 92 has a top surface formed in the shape of the second recess 83b, as shown in FIG. 16.

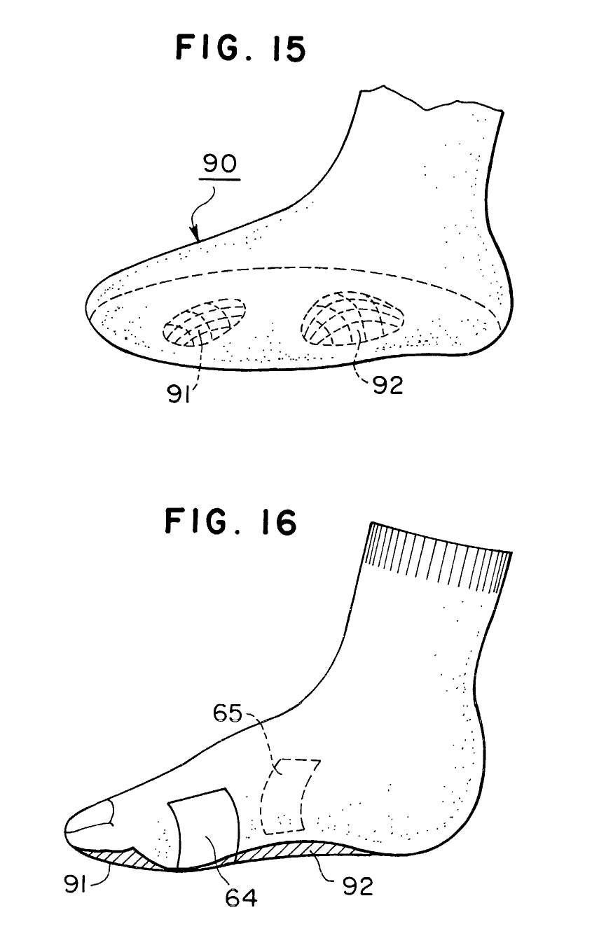

When a wearer puts her or his foot into the hose 90 having the first and second protuberant member 91 and 92, the first and second recesses 83a and 83b of the sole are pressed or kept by the first and second protuberant members 91 and 92 so that both or either of the first and second recesses 91 and 92 are positioned at the correct position, as shown in FIG. 15.

### EFFECTS OF THE INVENTION

As above described, when a wearer put her or his foot into a shoe in which the corrective appliance is attached, the foot excessively deformed or deviated by cause of Hallux Vulgus, is corrected or prevented, because the pressing (or reinforcing) members of the corrective appliance provided on the inner surface of the shoe at the frank thereof press the first finger or phalange and the first metatarsal caput that are in the area protruding the correct position, and the lowermost proximal end of the fifth metatarsal that are in the area position protruding form the correct position. Since the corrective appliance according to the present invention reduces the width of the wearer's footwear to press the protuberant areas of the foot, the first toe is not deformed or deviated, or returned from the deformed or deviated state to the correct position, whereby the force applied to the first toe or the first phalange is eliminated. Therefore, the corrective appliance allows for preventing or correcting excessive deformity or deviation of a foot as caused by unbalanced walking that a part of toes are not brought into contact with the ground, or the Hallux Vulgus affects. The corrective appliance thus will have as described below.

The foot has a dynamic mechanism such that the foot is returned from the deformed or deviated state to the corrected state, when the first toe or phalange and the first metatarsal caput that are in the area protruding form the correct position, and the lowermost proximal end of the fifth metatarsal that are in the area protruding form the correct position, are pressed. Since the corrective appliance according to the present invention presses the protuberant portions of the first toe or phalange and the first metatarsal caput, and the protuberant portion of the lowermost proximal end of the fifth metatarsal, the foot is returned from the deformed or deviated state to the corrected state so that distances between the toes are increased whereby the force is applied to the ground by the toes. Therefore, the wearer can walk, balancing on her or his toes. As a result, the corrective appliance according to the present invention allows for correcting or preventing a foot from being deformed or excessively deviated by cause of Hallux Vulgus or an unbalanced foot that a part of toes is not brought into contact with the ground.

Adopting the dynamic mechanism, the point where the force is applied (first toe), the supporting point (first metatarsal caput), and the point of application (lowermost proximal end of the fifth metatarsal), the corrective appliance is sure to provide increase in effects.

The corrective appliance according to the present invention allows for naturally correcting the deformed or deviated foot without excessively pressing the foot from the side thereof.

The corrective appliance according to the present invention is not used for the shoes to fit the sole of foot, but it is used for the shoes so that the foots obtains the corrected state whereby the sole of foot is balanced.

When the corrective appliance according to the present invention is used for a pair of hose, the looped elastic band or elastic bands having elasticity more than other cloth of a pair of hoses are circumferentialy arranged in the areas of the hoses, where the metatarsals will be brought into contact therewith at the time when a foot is put into the hose, and the pressing (or reinforcing) members are provided in the areas where the protuberant areas of the first toe or phalange and metatarsal caput, and of the fifth metatarsal will be brought into contact therewith at the time when a foot is put into the hose having the corrective appliance, whereby the deviation of the sole is corrected as a whole. In other words, the corrective appliance enables pressing the joint of the first toe or phalanx and metatarsal caput which are in the area protruding upward and obliquely rightward or leftward, into the correct position, while pressing the lowermost portion of the fifth metatarsal at the distal end thereof obliquely protruding outward into the correct position. Thereby, the first finger as being protruded by Hallux Vulgus, and the lowermost portion of the distal end of the fifth metatarsal as being protruded by Hallux Vulgus is easily corrected. That is, the foot is naturally returned from the deformed or deviated state to the corrected state. As a result, the wearer can walk correctly, balancing on whole soles.

In the case where both or one of the first protuberant member formed in the shape of the widthwise arch of the sole, and the second protuberant member formed in the shape of the lengthwise arch of the sole are additionally provided in the corrective appliance applied to the hose, the widthwise and lengthwise arches of the sole are corrected, whereby the wearer can walk, correcting her or his posture.

The above description was made in relationship with one foot having Hallux Vulgus. However, it should be understood that the present invention is not limited to the purpose for correcting one foot.

1. A corrective appliance provided on a footwear for correcting or preventing deformity or deviation of a sole or a foot: comprising first pressing means mounted inside of the footwear for pressing a first toe and a caput of a first metatarsal that are in the area protruding from a correct position, and second pressing means mounted inside of the footwear for pressing a lowermost proximal end of a fifth metatarsal which is in the area protruding from a correct position so that a deformed or deviated sole or foot is corrected, said first and second pressing means each having a front surface contacting with said first toe and said first metatarsal, and a back surface contacting with the inside surface of the footwear.

2. A corrective appliance provided on a footwear for correcting or preventing deformity or deviation of a sole or a foot as defined in claim 1, wherein said footwear comprises sandals each having a wide strap for covering phalanges to metatarsals, and said first and second pressing means are mounted on the inside surface of the wide strap.

3. A corrective appliance provided on a footwear for correcting or preventing deformity or deviation of a sole or a foot as defined in claim 1, wherein said footwear comprises Japanese sandals each having a clog thong provided on the top surface thereof which is in the area between a first toe and second toe, when a wearer's foot is put on the top surface, a strap extending from said clog thong to both side edges of the sandal, and a band provided under said strap, said band extending between the caput of the first metatarsal on the side of a distal end thereof and the lowermost proximal end of the fifth metatarsal, and said first and second pressing members are mounted on the inside surface of said band.

4. A corrective appliance provided on a footwear for correcting or preventing deformity or deviation of a sole or a foot as defined in claim 1, wherein said footwear comprises Japanese sandals each having a clog thong provided on the top surface thereof which is in the area between a first toe and second toe, when a wearer's foot is put on the top surface, a wide strap extending from said clog thong to both side edges, said wide strap extending between the first metatarsal and the fifth metatarsal, and wherein said first and second pressing members are mounted on the inside surface of said wide strap.

5. A corrective appliance provided on a footwear for correcting or preventing deformity or deviation of a sole or a foot as defined in claim 1, wherein said footwear comprises a shoe having an insole provided therein, and wherein said first pressing means is provided at the side edge of said insole on the side of the caput of the first metatarsal, and second pressing means is provided at the side edge of said insole on the side of the lowermost proximal end of the fifth metatarsal, said first and second pressing means protruding below the side edge of said insole, while contacting with the caput of the first metatarsal and the lowermost proximal end of the fifth metatarsal at the time when said insole is provided in a shoe.

6. A corrective appliance provided on a footwear for correcting or preventing deformity of deviation of a sole or a foot as defined in claim 1, wherein said footwear comprises hoses each having a first looped band for covering a sole and an instep which is in the area approximately extending from balls of phalanges at the proximal end thereof to the balls of the metatarsals at the distal end thereof, and a second looped band for covering the sole and the instep that are in the area approximately extending from the medial portion of the metatarsals to balls of the metatarsals at the proximal end, said looped band comprising elastic material having elasticity larger than other knitted area, and wherein said first pressing member, and second pressing member are mounted on said first looped band, and second looped band, respectively.

7. A corrective appliance as defined in claim 6, wherein said hoses has cushioning member provided inside thereof in face to face relationship with a heel of the wearer's foot.

8. A corrective appliance provided on a footwear for correcting or preventing deformity of deviation of a sole or a foot as defined in claim 1, wherein said footwear comprises hoses each having a looped band for covering a sole and an instep which is in the area approximately extending from balls of toes at the proximal end thereof to balls of toes at the proximal end, said looped band comprising elastic material having elasticity larger than other knitted area, and wherein said first and second pressing members are mounted on said looped band.

9. A corrective appliance as defined in claim 6, wherein said hoses has cushioning member provided inside thereof in face to face relationship with a heel of the wearer's foot.

10. A corrective appliance provided on a footwear for correcting or preventing deformity of deviation of a sole or a foot as defined in claim 1, wherein said footwear comprises hoses each having a first looped band for covering a sole and an instep which is in the area approximately extending from balls of toes at the proximal end thereof to the balls of the metatarsals at the distal end thereof, and a second looped band for covering the sole and the instep that are in the area approximately extending from the medial portion of the metatarsals to balls of the metatarsals at the proximal end, said looped band comprising elastic material having elasticity larger than other knitted area, and wherein said first and second pressing members are mounted on said looped bands, said corrective appliance further comprising one or both of a first protuberant member having a top surface formed in a shape of a widthwise arch of said sole for correcting the widthwise arch, and a second protuberant member having a top surface formed in a shape of a lengthwise arch of said sole for correcting the lengthwise arch.

11. A corrective appliance provided on a footwear for correcting or preventing deformity or deviation of a sole or a foot as defined in claim 1, wherein said footwear comprises hoses each having a looped band for covering a sole and an instep which is in the area approximately extending from balls of toes at the proximal end thereof to balls of toes at the proximal end, said looped band comprising elastic material having elasticity larger than other knitted area, and wherein said first and second pressing members are mounted on said looped band, said corrective appliance further comprising a first protuberant member having a top surface formed in a shape of a widthwise arch of said sole for correcting the widthwise arch, and a second protuberant member having a top surface formed in a shape of a lengthwise arch of said sole for correcting the lengthwise arch.

12. A corrective appliance provided on a footwear for correcting or preventing deformity or deviation of a sole or a foot as defined in claim 1, wherein said footwear comprises shoes, wherein said dressing members comprises a wall of said shoe, the wall having portions which is thickened relative to other portion to form said pressing members.
